(19) United States
(12) Patent Application Publication
(10) Pub. No.: US 2025/0288386 A1
(43) Pub. Date: Sep. 18, 2025

(54) STERILE FIELD SURGICAL IMAGING SYSTEM AND METHOD

(71) Applicant: Ocean Orthopedics, Inc., Westport, MA (US)

(72) Inventors: Thomas Gamache, Westport, MA (US); Raymond Parfett, Westport, MA (US); James Paiva, Westport, MA (US); Samuel Grossman, Westport, MA (US)

(21) Appl. No.: 19/082,813

(22) Filed: Mar. 18, 2025

Related U.S. Application Data
(60) Provisional application No. 63/566,537, filed on Mar. 18, 2024.

(51) Int. Cl. A61B 90/00

(52) U.S. Cl. CPC A61B 90/37; A61B 2090/372

(57) ABSTRACT

A surgical imaging system for communicating images from an imaging sensor to a display device within an operating room has a video controlling unit configured to receive the images from the imaging sensor. The video controlling unit is sterile or sterilizable sufficient for use in the sterile field of an operating room. The video controlling unit is configured to atraumatically rest on the patient during the procedure.

### PRIORITY

This patent application claims priority from provisional U.S. patent application No. 63/566,537, filed Mar. 18, 2024, entitled, “STERILE FIELD LIVE IMAGING,” attorney docket number 125381-10201, and naming Thomas Gamache, Raymond Parfett, James Paiva, Samuel Grossman, Christopher Ramsay, John Davis, and Kenneth Prada as inventors, the disclosure of which is incorporated herein, in its entirety, by reference.

### FIELD

Illustrative embodiments of the invention generally relate to medical devices and, more particularly, various embodiments of the invention relate to surgical imaging in a sterile field of an operating room.

### BACKGROUND

Image-guided surgery is a procedure in which a healthcare provider (e.g., anesthesiologist, physiatrist, physician assistant, nurse practitioner, or physician) uses real-time images of the body during surgery. These images are typically generated by cameras, X-rays, computers, or other imaging equipment. For many years, the primary technique in image-guided surgery was fluoroscopy, which produces an X-ray “movie” of the procedure in real time. Fluoroscopy remains a widely used and time-tested method in surgical imaging.

When providers need more than the 2D images produced by fluoroscopy, they have advanced alternatives. Newer technologies offer detailed 3D guidance, enhancing precision during surgery. These systems, known by various trademarked names, function like a GPS for the operating room. They track surgical instruments in real time, transmitting their position to a display device that displays their approximate location inside the body.

Image-guided surgery is sometimes used in traditional open procedures and is commonly employed in minimally invasive spinal surgery. In minimally invasive surgery, a provider makes small skin incisions and uses minimal retraction to access the surgical field. Specialized instruments minimize tissue disruption, allowing surgical tools to reach the target area without the need for extensive exposure. Compared to traditional surgery, minimally invasive techniques can reduce tissue damage, post-surgical pain, and complications while promoting faster recovery. However, because the surgical area and instruments may not be visible to the naked eye, image-guided surgery—particularly with contemporary 3D navigation systems—has been instrumental in advancing minimally invasive techniques for both adult and pediatric patients.

The images from CT and MRI scans function like the pre-loaded “map” in a GPS navigation system. These scans are uploaded to a computer and converted into 3D images. Before surgery, the provider can zoom in on specific areas of interest, viewing them from multiple angles and perspectives for enhanced precision.

In spinal surgery, image guidance typically begins with a CT or similar scan performed in the operating room to generate the most up to date “map” of the body. Special cameras track the surgical instruments in real time, while the computer integrates the CT scan with the instrument's location data. This combined visualization is then displayed on a screen, allowing the provider to navigate the procedure with enhanced precision.

### SUMMARY OF VARIOUS EMBODIMENTS

In accordance with one embodiment of the invention, a surgical imaging system for communicating images from an imaging sensor to a display device within an operating room has a video controlling unit configured to receive the images from the imaging sensor. The video controlling unit is sterile or sterilizable sufficient for use in the sterile field of an operating room.

The video controlling unit has functional components that are specially configured to produce improved results. Among others, those include a video processor configured to alter the received images to produce processed images from the received images. Preferably, the video processor is configured to produce the processed images in a protocol that is compatible with the display device for real-time or near real-time display.

The video controlling unit also has a transmitter configured to wirelessly and substantially lag-free transmit the processed images from the sterile field to the display device. To that end, the transmitter is configured to transmit the processed images with an effective radiated power of 1-100 milliwatts at a frame rate compatible with a frame rate displayable by the display device (e.g., 30-60 frames per minute). A DC interface configured to receive direct current power powers both the transmitter and the video processor. The video controlling unit further has a housing containing at least in part one or more of the video processor and the transmitter. The housing also has at least one patient contact surface formed from a biocompatible material sized to be supported on the patient's body in the sterile field when imaging with the imaging sensor. The video controlling unit configured to cause no trauma to the patient when supported on the patient's body.

Among other things, the protocol may comprise High-Definition Multimedia Interface. Moreover, the display device may be positioned outside of the sterile field and within the operating room. Alternatively, the display device may be in the sterile field (e.g., a LED panel, or a wireless headset worn by a physician or other user). The imaging sensor may use any of a variety of modalities, such as a camera using one or more of IR, visible, UV and ICG wavelengths. In fact, an inertial measurement unit may be coupled with the imaging sensor and producing one or more of angular velocity, acceleration, position, and orientation signal relating to inertial activity of the sensor.

To facilitate the surgical procedure, the video controlling unit is sized to have a low profile to avoid obstructing surgical access when on the patient's body. For example, the video controlling unit may have a length, width, and height, with the length being 3 to 9 inches, the width being 3 to 14 inches, and the height being 3 to 8 inches. The video controlling unit may have a weight of 8 to 32 ounces.

Since it is in the sterile field, the video controlling unit may have an input for receiving input information from a user that at least in part controls the video controlling unit. For example, the input may be one or more of a tactile input, an input configured to receive voice commands, or an input configured to receive gesture commands. In some embodiments, the video processor is configured to alter the received images by changing one or more of brightness, a contrast, a sharpness, a white balance, a black level, a zoom level, apply a color correction, or rotation.

To ensure sanitization before use, the video controlling unit may be sealed in an air-tight package having a sanitized interior. Before use, the video controlling unit may be removed from the package (e.g., in the sterile field). In a similar manner, the housing may be sealed to have a liquid resistant interior containing the video processor. To facilitate use, the system also may have a computer program product (for use on a computer system) including a tangible, non-transient computer usable medium having computer readable program code thereon. The computer readable program code may include program code for controlling the video controlling unit.

In accordance with another embodiment, a surgical imaging system communicates images from an imaging sensor to a display device within an operating room and has a battery powered video controlling unit configured to receive the images from the imaging sensor. The video controlling unit is sterile sufficient for use in the sterile field of an operating room. The video controlling unit has a video processor configured to alter the received images to produce processed images from the received images in a display protocol of the display device, and a transmitter configured to wirelessly and substantially lag-free transmit the processed images from the sterile field to the display device for real-time display or near real-time display. The transmitter also is configured to transmit the processed images with the display protocol toward the display device at a power at or between about 0.5 meters to 50 meters away from the transmitter.

A housing contains at least in part one or more of the video processor and the transmitter. The housing has at least one patient contact surface formed from a biocompatible material and sized to be supported on the patient's body in the sterile field when imaging with the imaging sensor. In preferred embodiments, the video controlling unit is configured to cause no trauma to the patient when supported on the patient's body. To enable control, the video controlling unit also has an input for receiving input information from a user via one or more of user voice, user gesture, and direct user contact. The video controlling unit can be controlled at least in part by the received input information.

In accordance with another embodiment, a method of imaging during a surgical procedure for a display on a display device that communicates and/or operates in accordance with a display protocol positions an imaging sensor relative to a patient in the surgical field of an operating room to produce a plurality of images of the patient during the surgical procedure. The method images the patient during the surgical procedure using the imaging sensor, places a sterile video controlling unit directly on the patient during imaging so that the patient's body is supporting the video controlling unit without causing trauma to the patient's body, and powers the video controlling unit in the sterile field with DC power either before or after positioning on the patient's body.

The method receives, via the video controlling unit, images generated by the imaging sensor, alters the received images to produce processed images in the display protocol from the received images. The method then transmits wirelessly, substantially lag-free, and with an effective radiated power of 1-100 milliwatts, the processed images from the sterile field to the display device at a frame rate compatible with a frame rate displayable by the display device. Accordingly, the display device displays the processed images in real-time or near real-time.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows an operating room that may implement illustrative embodiments.

FIG. 2 schematically shows sterile field imaging system in accordance with illustrative embodiments.

FIG. 3 schematically shows an operating room highlighting the sterile field and the relationship with the sterile field imaging system in accordance with illustrative embodiments.

FIG. 4 schematically shows more detail s of the sterile field imaging system, including details of a sensor/camera, in accordance with illustrative embodiments.

FIG. 5 schematically shows a sterile field imaging system, with more details of a video controlling unit in accordance with illustrative embodiments.

FIG. 6 shows a process of preparing and using the sterile field imaging system to obtain video or image information in accordance with illustrative embodiments.

FIG. 7 shows a video processing process for the sterile field imaging system in accordance with illustrative embodiments.

### DESCRIPTION OF ILLUSTRATIVE EMBODIMENTS

Illustrative embodiments facilitate display of video and images from a surgical camera while, in many cases, reducing capital costs. Specifically, video processing can be substantially completed right in the sterile field of an operating room during a surgical procedure. To that end, a sterile video controlling unit is specially configured to process images and video and, optimally, direct physician control from the sterile field. Details of illustrative embodiments are discussed below.

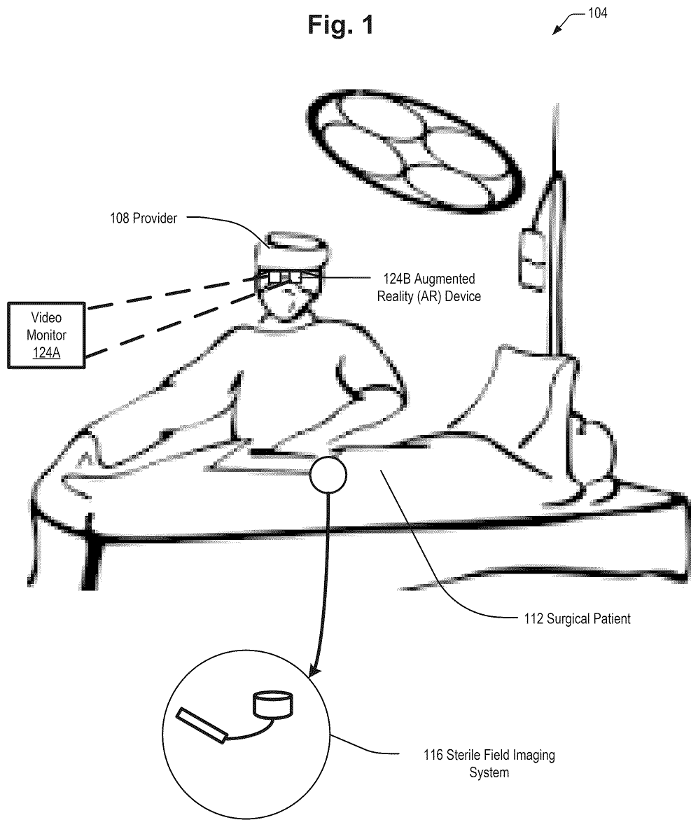

FIG. 1 schematically shows an operating room 104 using a sterile field imaging system in in accordance with illustrative embodiments. Many items not shown in FIG. 1 may or may not be required in an operating room 104 or surgical suite. As such, FIG. 1 is simplified and not intended to be exhaustive. To demonstrate various embodiments, FIG. 1 shows a back surgery procedure as an example, although illustrative embodiments are not limited to any specific procedure or procedures. Among other things, the operating room 104 may include an operating table for a user or provider 108 (e.g., a doctor/physician) to conduct a surgical procedure on a patent 112.

As shown in FIG. 1, the provider 108 may use one or more nearby displays (aka “display devices” and generically identified by reference number 124) to display high-resolution video of the specific area being operated upon (operating field). In many cases, the specific area may be magnified to show a maximum amount of detail where the interaction between surgical instruments and body areas is important to the procedure. The display devices 124 may include one or more monitors 124A acting as display devices 124 that display images from one or more cameras (also referred to as “sensors”), as discussed herein. In one embodiment, the display device 124 may include an augmented reality (AR) device or eyewear, such as AR glasses 124B, worn by the provider 108. The AR device 124B may allow a conventional view of whatever the provider 108 sees, while overlaying text and/or synthetic images that assist the provider 108 in identifying what the camera is presenting. Examples of an AR device 124B or mixed-reality wearable display may include devices distributed under the trade names HOLOLENS, OCULUS, or a VISION PRO. Some display devices 124 may be located outside the sterile field (e.g., a video monitor 124A), while others may be located inside the sterile field (e.g., the AR device 124B and/or the monitor/display device 124A).

FIG. 1 also shows an enlarged view of a representative sterile field 308 imaging system (hereinafter “imaging system 116”) located within a sterile field of the operating room 104. The imaging system 116 performs functions related to imaging for the procedure and processing the imaged video/pictures for display by the display device(s) 124.

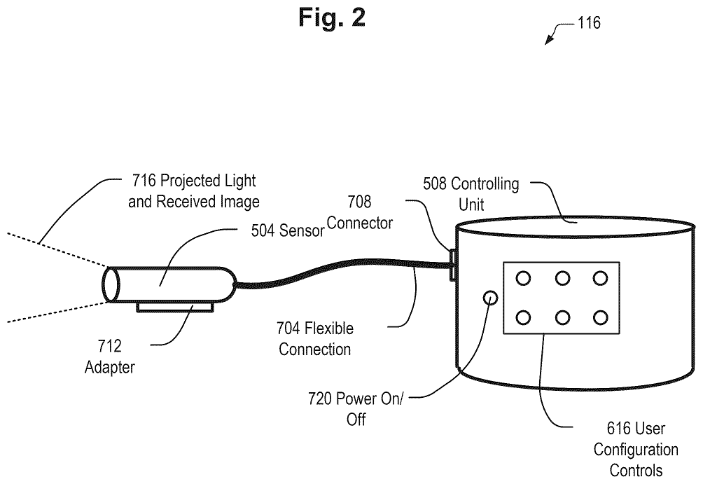

More particularly, FIG. 2 schematically shows more details of the imaging system 116 in accordance with illustrative embodiments. As shown, the imaging system 116 may include a sensor 504 (e.g., a camera, also identified by reference number “504”) to capture images on or within the patient, in communication with and connected with a video controlling unit 508 (sometimes referred to for simplicity as “controlling unit”) by a flexible connection 704. Among other things and as discussed below, the video controlling unit 508 has hardware and software configured to receive image information from the camera, process the received image information, and forward the processed image information in an appropriate format (e.g., using a prescribed display protocol and/or transmission protocol) for display by the display device(s) 124.

The flexible connection 704 may removably connect to the controlling unit 508 through a connector 708. The connector 708 therefore is configured to enable the controlling unit 508 to be separated and reused, while the sensor 504 and flexible connection 704 may be discarded following a surgical procedure. Rather than use the tangible flexible connection 704, however, some embodiments may use a wireless connection channel to communicate the sensor 504 with the controlling unit 508.

Importantly, the controlling unit 508 is configured to be supported and rest on the patient during the surgical procedure. Specifically, the video controlling unit 508 preferably is configured to cause no trauma (e.g., negligible trauma) to the patient 112 when supported on the patient's body. To do this more effectively, the controlling unit is configured to be lightweight (e.g., 8 to 30 ounces), compact, and non-invasive, ensuring it can be safely placed directly on the patient 112 without causing any trauma. For example, the length may be 3 to 9 inches, the width may be 3 to 14 inches, and the height may be 3 to 8 inches. As such, its form factor allows it to rest gently on the skin (or on a sheet or blanket covering the skin) without exerting significant pressure, preventing meaningful discomfort or interference with the patient's physiology. Therefore, the video controlling unit 508 base optionally may incorporate soft, biocompatible materials or low-friction surfaces, further ensuring that contact with the body remains gentle and does not cause irritation or abrasions. A minimal weight distribution further reduces any risk of pressure-related effects, making it suitable for extended placement during a procedure. Additionally, the video controlling unit's edges may be rounded or tapered to prevent localized pressure points, ensuring that no sharp or rigid elements create undue force on the patient's body.

Since the video controlling unit 508 does not penetrate the skin, compress underlying tissue significantly, or interfere with circulation, the contact it makes with the patient 112 results in negligible trauma, meaning there is no lasting impact, tissue damage, or functional impairment. Negligible trauma—such as brief surface contact or minor pressure—does not meaningfully disrupt the body's natural state and is functionally equivalent to no trauma, as it does not introduce non-negligible pain, inflammation, or structural changes. This ensures the video controlling unit 508 can be confidently placed on the patient 112 during surgery without the risk of post-procedural effects, making it a safe and unobtrusive component of the surgical environment. Its design prioritizes patient safety, stability, and ease of use while maintaining full functionality in the sterile field 308 of the operating room 104.

In a corresponding manner, the video controlling unit 508 preferably is sized with a low profile, ensuring it does not obstruct or interfere with surgical access when resting on the patient's body. In a surgical setting, where precision and accessibility are paramount, every piece of equipment must be carefully designed to avoid interfering with the surgeon's workspace. A low-profile form factor allows the video controlling unit 508 to remain unobtrusive, minimizing impact on the surrounding sterile field 308. Its compact dimensions ensure that it does not create unnecessary bulk, allowing surgeons and medical staff to maintain clear visibility and unimpeded access to the operative site. This careful sizing also prevents the video controlling unit 508 from interfering with surgical instruments, personnel movements, or the overall ergonomic flow of the procedure, ensuring that critical tasks can be carried out efficiently without disruption.

Additionally, the low-profile nature of the video controlling unit 508 enhances stability and adaptability, allowing it to generally conform to different anatomical surfaces without protruding in a way that could cause unintended shifting or imbalance. A streamlined, contoured design may enable the video controlling unit 508 to rest securely on the patient 112 without requiring additional fixation mechanisms, further simplifying its integration into the surgical workflow. Because it does not extend significantly above the patient's body, the risk of accidental contact from surgical staff, tools, or drapes is minimized, reducing the chances of displacement or obstruction. This allows the video controlling unit 508 to function as intended while physically remaining a passive, non-intrusive component of the surgical environment.

Furthermore, material selection and weight distribution play key roles in ensuring the video controlling unit 508 remains low-profile while maintaining effectiveness. As previously noted, lightweight construction prevents unnecessary downward force on the patient's body, while smooth, rounded edges eliminate any risk of discomfort or pressure points. The combination of a slim, stable structure and ergonomic contouring ensures that the video controlling unit 508 can be placed and repositioned effortlessly, without interfering with ongoing surgical tasks. By prioritizing a compact, non-intrusive design, the video controlling unit 508 seamlessly integrates into the operating room 104, providing necessary functionality without compromising access, visibility, or patient comfort.

The video controlling unit 508 embodiment shown in the figures has a cylindrical form factor. Of course, other form factors may be utilized and still be lightweight, unobtrusive, and suitable for placement on the patient 112 during surgery. Alternative designs may include a low-profile, disc-shaped unit, which can rest securely on the patient 112 without causing discomfort, or a small, dome-like structure, allowing for stable placement while minimizing obstruction. Other configurations include a rectangular or wedge-shaped design, which could conform more naturally to different anatomical surfaces, or a shallow, puck-like form factor, ensuring even weight distribution for gentle positioning. Additionally, the video controlling unit 508 could feature a contoured base to better adapt to body contours, or a modular, multi-surface structure that allows flexibility in placement while maintaining stability throughout the procedure.

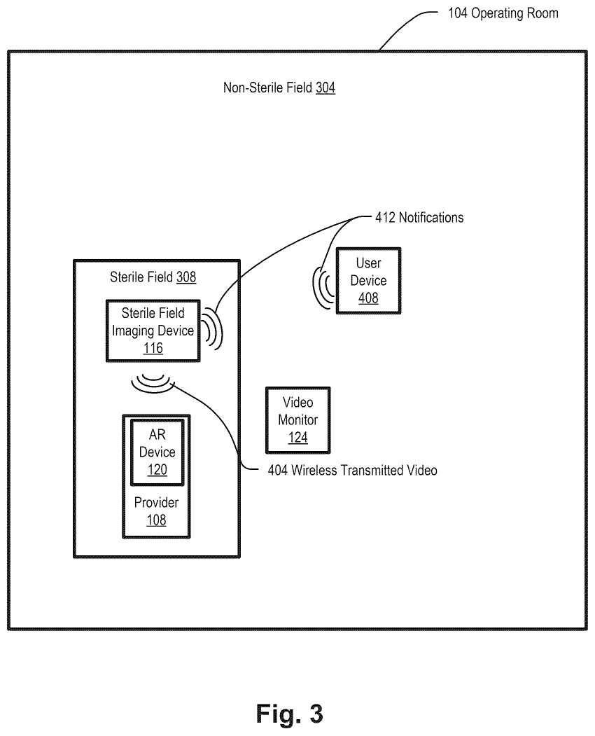

The sensor 504 is configured to project light and, in response, receive an image 716 through one end (i.e., the end away from the flexible connection 704 and the controlling unit 508). Therefore, stable mounting of the sensor 504 is useful to view a stable video image. To that end, the sensor 504 may be configured to interface with one or more adapters 712 to couple the sensor 504 to various other structures within the sterile field 308 (see FIG. 3 for more details regarding the sterile field 308). For example, adapter(s) 712 may couple the sensor 504 to retractors, stylettes, probes, and cannulas. In some embodiments, the adapter(s) 712 couple to an arm that statically or movably holds the sensor 504.

The controlling unit 508, which processes and prepares images received from the sensor 504 for display on the display device 124, has an outside housing with various controls, interfaces, and indicators on its outside surface. Favorably, these features enable easy access by operating room medical personnel in the sterile field 308. To that end, the controlling unit 508 illustratively has a power ON/OFF control 720 that powers up the sterile field imaging device/system 116, and a corresponding indicator (e.g., an LED—light emitting diode) that is lit when the imaging system 116 is powered ON. The controlling unit 508 may also have a fault indicator (e.g., an LED) that indicates a fault within the imaging system 116. The controlling unit 508 of various embodiments also has one or more indicators that show a current DC power level remaining in the system 116. For example, the controlling unit 508 may provide a group of LEDs in a ladder display that reflect a level of power expressed as a percent, a reliable runtime, or another metric.

In one embodiment, the controlling unit 508 may also include user configuration or use controls 616 (or other controls). Each parameter reflected in the configuration controls 616 may have “up” and “down” physical or virtual buttons to increase or decrease, respectively, a specific parameter. Parameters that have associated controls within the user configuration controls 616 may include a wide range of image and video adjustments to enhance the viewing experience. These controls can include brightness, contrast, sharpness, color or gamma correction, white balance, and black level. Additionally, users may have access to advanced settings such as image rotation, zoom levels, hue, saturation, noise reduction, edge enhancement, backlight compensation, dynamic range adjustment, exposure control, frame rate adjustment, deinterlacing, color temperature settings, aspect ratio control, and motion smoothing. Other controls may include local dimming, tone mapping, HDR toggle, grayscale mode, night mode, skin tone enhancement, and lens distortion correction, providing extensive customization for optimal video quality based on user preferences and environmental conditions.

The video controlling unit 508 may have a more versatile input interface designed to enhance usability by accommodating multiple interaction methods. For example, in addition to the discussed buttons, the video controlling unit 508 may incorporate tactile inputs, such as capacitive or piezoresistive touch surfaces, pressure-sensitive pads, or haptic-responsive zones, allowing for seamless and intuitive control. These inputs can provide users with responsive feedback, ensuring precise adjustments and interaction even in environments where traditional buttons may be less effective. The integration of such tactile options allows for greater flexibility in user interaction, enabling operation through light touches, swipes, or customizable pressure-based commands, depending on the context of use.

Beyond tactile inputs, the video controlling unit 508 may also feature voice and gesture recognition capabilities, expanding its accessibility and hands-free usability. A built-in microphone array or external voice command integration could allow users to control the video controlling unit 508 through spoken instructions, improving convenience in scenarios where physical interaction may be impractical or undesirable. Additionally, a gesture-based input system using infrared, ultrasonic, or camera-based tracking could enable users to interact with the video controlling unit 508 through simple hand movements, swipes, or proximity-based controls. This multimodal approach to input ensures that the video controlling unit 508 remains adaptable to different use cases, environments, and user preferences, providing a streamlined and efficient control experience that enhances both accessibility and operational ease.

In addition to tactile, voice, and gesture inputs, the video controlling unit 508 may support several other methods of input to enhance versatility and adaptability across different use cases. One such method could be proximity-based interaction, where the video controlling unit 508 detects a user's presence or hand movements through radio frequency identification (RFID), near-field communication (NFC), or Bluetooth Low Energy (BLE) signals, enabling seamless authentication or automatic activation without physical contact. Additionally, biometric input such as fingerprint scanning or facial recognition could be incorporated to provide secure, user-specific controls, particularly for applications requiring authentication or personalized settings. Finally, the video controlling unit 508 could include environmental input sensors, such as light, temperature, or pressure sensitivity, to trigger automated responses or adjustments based on real-time conditions, further enhancing the user experience through intelligent, context-aware interactions. By supporting multiple input methods, the video controlling unit 508 ensures maximum flexibility, accessibility, and ease of use across a wide range of applications.

The imaging system 116 also may support parameter configuration through an application of computer program code on the user device (e.g., smartphone, a tablet, laptop, personal computer, or other computing device). This form of “soft configuration” may be implemented either instead of or in addition to the user configuration controls 616. To support this form of soft configuration, the controlling unit 508 may include an alternate wireless transceiver 660 that may include a bidirectional wireless or wired interface, such as Bluetooth.

FIG. 3 schematically shows a different representation of the operating room 104 and imaging system 116. As shown, the operating room 104 includes the prior noted sterile field 308, and the prior noted non-sterile field 304, which is the area outside of the sterile field 308. More specifically, as known by those in the art, the sterile field 308 in an operating room 104 is a designated area that is meticulously maintained to be free from microorganisms, ensuring a contamination-free environment for the surgical procedure. It includes the surgical site, the instruments, the draped patient 112, and any medical personnel directly involved in the procedure who are properly scrubbed in. The sterile field 308 typically is established and maintained by adhering to strict aseptic techniques, such as surgical hand scrubbing, wearing sterile gowns and gloves, and using sterilized surgical instruments and drapes. The surgical team-including the surgeon, surgical assistants, and scrub nurses-must remain within this field and avoid contact with anything non-sterile to prevent contamination.

To maintain sterility, all items introduced into the field must be pre-sterilized, and any breach—such as accidental contact with a non-sterile object—requires immediate corrective action, such as replacing contaminated gloves, instruments, or drapes. The sterile field 308 may or may not be defined by surgical drapes, which create a barrier between the sterile and non-sterile areas. Sterile team members should only interact with sterile objects, and their hands must remain above the waist and within the sterile field 308 at all times to prevent cross-contamination. The sterile field 308 plays a critical role in preventing surgical site infections (SSIs), ensuring patient safety, and maintaining the integrity of the procedure. The sterile field 308 also typically does not include AC power receptacles.

In contrast, as noted above, the non-sterile field 304 includes areas and personnel in the operating room 104 that are not part of the sterile field 308. This includes circulating nurses, anesthesiologists, surgical lights, monitoring equipment, and surfaces such as walls, floors, and furniture. These elements play essential supporting roles but are not directly involved in the sterile aspects of surgery. For example, circulating nurses handle instrument preparation, adjust settings on medical devices, and pass sterile items into the field without contaminating them, ensuring the smooth operation of the procedure while avoiding direct interaction with sterile materials.

Additionally, the anesthesiologist and other support staff typically work outside the sterile field 308 to manage patient monitoring, anesthesia administration, and equipment operation. While they maintain high hygiene standards, they do not need to adhere to the same strict sterility rules as the surgical team. A clear separation is maintained between the sterile and non-sterile areas 308 and 304 to prevent accidental contamination. Any individual moving between these areas 304 and 308 must follow specific protocols, such as wearing appropriate protective gear, passing sterile instruments in a controlled manner, and maintaining a safe distance from the sterile field 308. The non-sterile field 304 is essential for operational support, equipment handling, and overall patient care, working in tandem with the sterile field 308 to ensure a safe and successful surgical procedure.

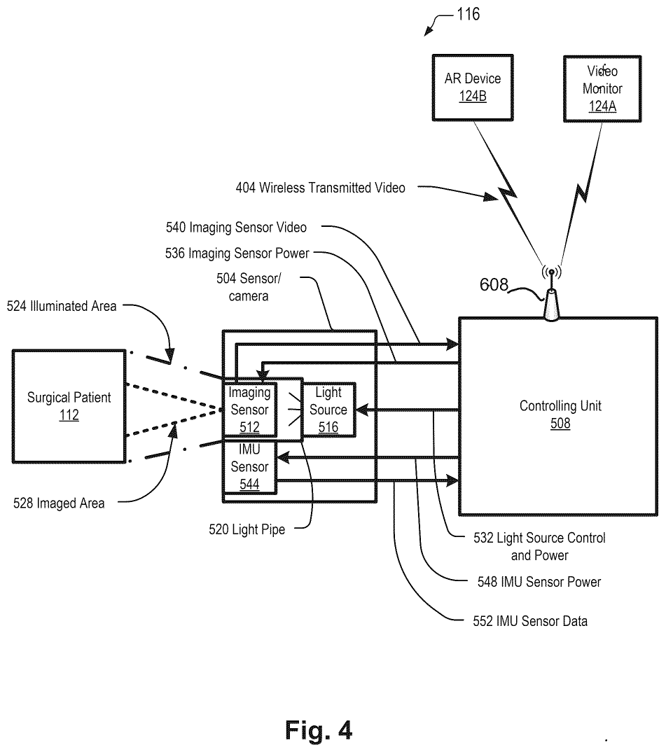

In preferred embodiments, instruments in the sterile field 308 may include the imaging system 116, which includes the sensor 504 and the video controlling unit 508. In one embodiment, the video controlling unit 508 may include a video cable (e.g., HDMI cable) that extends to a display device 124 in the non-sterile field 304. In a preferred embodiment, however, the video controlling unit 508 is configured to wirelessly transmit video 404 (FIG. 4) to the display device 124 in the non-sterile field 308, or to the AR device 124B worn by the provider 108. This embodiment beneficially does not require any wires between the sterile field 308 and the non-sterile field 304. An AR device 124B may be preferable to one or more monitors 124A because the AR device 124B allows the provider's vision to be concentrated on the portion of the patient 112 and the surgical instruments within the sterile field 308. Watching a monitor 124A may force the provider 108 to look away from the operating field and the patient 112.

In one embodiment, the imaging system 116 may transmit and receive notifications 412 to and from, respectively, one or more user devices 408. User devices 408 may include a computing device associated with one or more operating room personnel. User devices 408 may include smart phones, smart watches, tablets or iPads, desktop computers, servers, wearable computers, and the like.

FIG. 4 schematically shows more details of the sensor/camera 504 in accordance with illustrative embodiments. This embodiment separates the lighting and camera parts of the imaging device from its computing (e.g., the controlling unit 508), power supply, and communication portions. However, in other embodiments, more or all components of the imaging system 116 could be integrated into a single assembly.

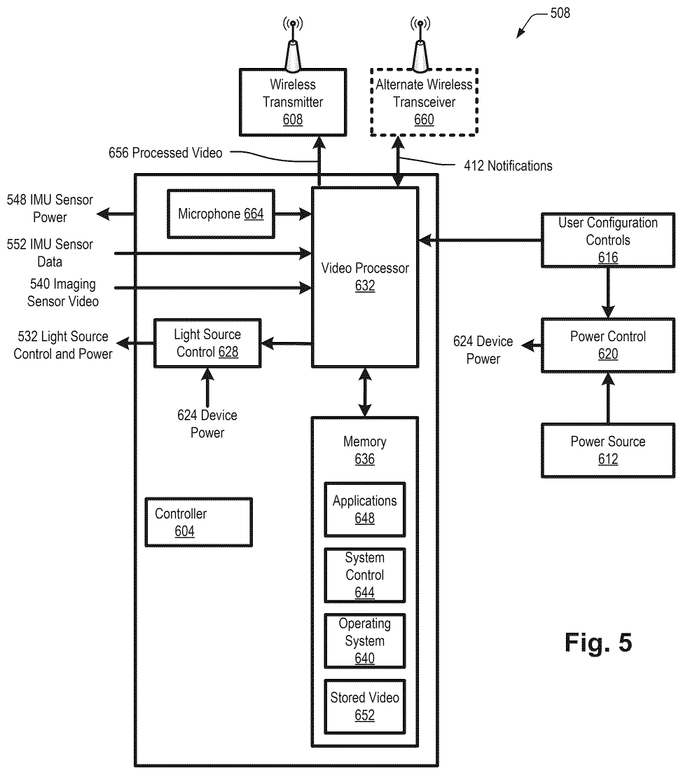

As shown in FIGS. 1 and 2, the imaging system 116 may be organized as three primary components: the sensor 504, the controlling unit 508, and the flexible connection 704 between the sensor 504 and the controlling unit 508. Each is discussed immediately below with respect to FIGS. 4 and 5.

The sensor 504 includes at least an outer housing, an imaging sensor 512 to image the patient 112, and a light source 516. In one embodiment, the imaging sensor 512 may be a 720×720-pixel sensor producing imaging sensor video 540 of 30-60 frames/second. As it may be used in tight physiologic areas, the face of the imaging sensor 512 preferably is small. For example, the face of the imaging sensor 512 may be approximately one millimeter (mm) square. The imaging sensor 512 may have a corresponding field of view that produces an imaged area 528.

The sensor 504 may include a device to apply a controlling fluid that passes over the imaging sensor 512 to provide continuous cleaning. In another embodiment, a removable cover (e.g., tear-away cellophane roll) may be initially applied to the imaging sensor 512. In another embodiment, water droplets on the imaging sensor 512 may be utilized to allow for lensing and correction of imaging sensor video 540 due to lensing. In another embodiment, the sensor 504 may include a feature that covers the imaging sensor 512 during introduction into a body and is later removed.

The light source 516 may be LEDs, miniature light bulbs, or other forms of light sources 516. In one embodiment, the light sources 516 may be located around the imaging sensor 512 near a front surface of the imaging sensor 512 (not shown). This may have the disadvantage of producing heat in front of the imaging sensor 512, which may be medically undesirable. This may also make the sensor 504 wider since the light sources 516 contribute to the cross-section profile.

In a preferred embodiment, the light source or sources 516 may be located behind the imaging sensor 512. In this configuration, a light pipe 520 may surround the imaging sensor 512 and convey light from the light sources 516 through the light pipe 520 and project an illuminated area 524 within the surgical patient 112. The light pipe 520 may also provide a benefit of diffusing light throughout the illuminated area 524, resulting in more uniform lighting. The sensor 504 and included components may be sterilizable and/or disposable and may be distributed as sterilized components in suitable packaging.

To further functionality, the sensor 504 also may include an inertial measurement sensor (IMU) 544. The IMU sensor 544 is an electronic device, typically an integrated circuit, that measures and reports angular velocity, acceleration, position and/or orientation of the imaging sensor 512, using one or more of accelerometers, gyroscopes, magnetometers, or the like. In one embodiment, the IMU may include a wireless inertial measurement sensor (WIMU). The controlling unit 508 provides IMU sensor power 548 to the IMU sensor 544 and the IMU sensor 544 provides IMU sensor data 552 to the controlling unit 508.

The IMU 544 beneficially may track camera positions for malpractice, teaching residents (telestration), and data collection/data mining for improved outcomes and continuous learning applications. The IMU 544 may aid spatial computing and motion tracking of tools and implants to better quantify outcomes and analyze provider fatigue.

Alternatively, a flex circuit may provide the electrical connections between the sensor 504 components and the controlling unit 508 components. As such, the flex circuit interconnects the imaging sensor 512, the light source 516, the IMU sensor 544 (if present), and the controlling unit 508. In another embodiment, the electrical connections may be provided by conventional wiring surrounded by a sterilizable jacket. The electrical connections may include imaging sensor power 536, imaging sensor video 540, light source control and power 532, IMU sensor power 548, and IMU sensor data 552. In one embodiment, the electrical connections may be separated from the controlling unit 508 by a connector affixed to an outside wall of the controlling unit 508. This may allow the sensor 504 with flexible connections to be modularly separated from the controlling unit 508 and disposed while the controlling unit is sanitized and repackaged, following a surgical procedure. In one embodiment, the flexible connection may include one or more light sources 516 or user controls 616.

Also as shown in FIG. 4, the video controlling unit 508 includes a wireless transmitter 608 that may bidirectionally communicate with external devices, such as computers (e.g., tablets, laptops, smartphones, not shown in FIG. 4 but shown in other figures) and display devices 124. In preferred embodiments, the transmitter 608 and video controlling unit 508 are configured to generate and send a simple yet high-quality signal, ensuring that the processed images from the sterile field 308 are efficiently transmitted to the display device 124 with minimal delay. The video controlling unit 508 processes the image data quickly and optimizes it for transmission, ensuring that the signal remains clear, stable, and synchronized with real-time surgical actions. This efficient processing minimizes buffering or lag and may eliminate encryption (i.e., use no encryption), allowing the transmitted images to reflect the actual state of the surgical field without perceptible delay to a typical human viewer. The transmitter ensures that the signal remains strong and interference-free, maintaining a seamless connection between the sensor 504 and the display device 124.

To achieve substantially lag-free performance, the system may use standardized formats such as HDMI, DisplayPort, or similar digital video transmission protocols, which allow for high-resolution video to be sent with minimal compression and near-instantaneous rendering. By maintaining a direct, streamlined video pipeline, the transmitter reduces the risk of artifacts, frame drops, or signal degradation. The system this is optimized to process and transmit data efficiently, ensuring that the display device 124 receives a faithful, real-time representation of the surgical field, enhancing visualization without introducing unnecessary complexity or latency. Indeed, in the real world, while there may be glitches and imperfections, this real-time display should provide satisfactory results.

In real-time surgical imaging, maintaining a synchronized frame rate between the transmission system and the display device 124 is important for accurate visual representation. Accordingly, the video controlling unit 508 and transmitter preferably are configured to produce processed images at a frame rate compatible with the display device 124, ensuring smooth and natural motion with minimal or no stuttering, lag, or dropped frames. The transmitter 608 and controlling unit 508 thus operate in a manner that is compatible with the display device 124. For example, they may process images at standard frame rates, typically 30, 60, or even 120 frames per second (FPS), depending on the capabilities and protocols of the display device 124 and the required level of detail. By ensuring that the transmitted frame rate precisely matches what the display device 124 can process, the system eliminates issues such as frame mismatching, tearing, or ghosting, which can degrade the clarity and responsiveness of the visuals. This synchronization allows the surgical team to receive real-time feedback without visual artifacts, ensuring that what is seen on the display device 124 precisely aligns with the actions being performed in the sterile field 308.

In addition to frame rate compatibility, the controlling unit 508 and transmitter are preferably configured to operate with an effective radiated power (ERP) of 1-100 milliwatts, striking a balance between signal strength, efficiency, and regulatory compliance. This power range is sufficient to ensure a stable, interference-free wireless connection while remaining within safety limits to prevent excessive electromagnetic emissions in a sensitive medical environment. A lower ERP, such as 1-10 milliwatts, may be used in smaller, enclosed operating rooms 104 where short-range, high-bandwidth communication is prioritized, minimizing potential interference with other medical equipment. For larger surgical suites or environments requiring greater transmission range and penetration, the transmitter can operate at higher power levels within the 100-milliwatt limit, ensuring that the signal maintains integrity without excessive power consumption or interference with surrounding wireless systems.

By optimizing both frame rate synchronization and transmission power, the imaging system 116 ensures that the display device 124 receives high-quality, real-time surgical images without noticeable distortion or lag. Efficient processing and wireless communication protocols by the controlling unit and transmitters ensure that image clarity, motion smoothness, and transmission stability are maintained, providing the surgical team with precise, uninterrupted visualization throughout the procedure. These design considerations contribute to a seamless, responsive imaging experience, ensuring that the technology enhances surgical precision without introducing delays, distortions, or operational inefficiencies.

Accordingly, the controlling unit and transmitter are configured to produce and transmit processed images using one or more transmission and/or operational protocols that are compatible with the display device 124, ensuring seamless, real-time or near real-time display. The video controlling unit 508 processes the image data efficiently, encoding it into a widely used video transmission format such as HDMI, DisplayPort, or a wireless video transmission standard like Wi-Fi 6 or WHDI (Wireless Home Digital Interface). These protocols support high-resolution, high-frame-rate video streams with minimal compression, preserving image quality while maintaining a low-latency signal path. Alternatively, lower quality protocols also may suffice if the application permits it.

On the display device side, the video protocol must be correctly interpreted and rendered in real-time without buffering or processing delays. The display device 124 operates using a frame synchronization protocol, ensuring that the received video stream is displayed at the correct refresh rate—typically 30, 60, or 120 Hz—without mismatches that could cause flickering or misalignment. Additionally, color space compatibility (such as sRGB, DCI-P3, or Rec. 709) ensures accurate color reproduction, which is essential for distinguishing tissue variations and surgical details. The combination of optimized transmission protocols and display synchronization allows for fluid, lag-free visualization of the sterile field 308, enabling the surgical team to make precise, real-time decisions based on a faithful representation of the operative environment.

As also shown in earlier discussed FIG. 2, the components within the sensor 504 may be rigidly seated within a housing/sensor body. In one embodiment, a sensor 504 (FIG. 4) may be removably coupled to a fixed locator that maintains an original position relative to the patient 112. The fixed locator may be a clip that is coupled to a cannula. In one embodiment, the provider 108 may detach the sensor 504 from the clip to clean or shift the imaging sensor 504 view while the clip remains in-place to maintain a standard view location. The seating may be performed by one or more O-rings, or a friction fit between the components 512, 516, 520 and an inner wall of the sensor 504 housing. In one embodiment, the components 512, 516, 520, and 544 may be encapsulated in a suitable epoxy either prior to or after seating the components within the sensor body. In another embodiment, the sensor 504 may be significantly smaller than an endoscope. This may allow the imaging sensor 512 and light source 516 to “see around” skin and internal body structures that a rigid rod of many types of arthroscopes and/or endoscopes may not allow.

FIG. 5 schematically shows more detail of the controlling unit 508 in accordance with illustrative embodiments. As shown, the controlling unit 508 preferably has a housing that contains many of the noted components. In illustrative embodiments, the housing is one or more of liquid-tight, air-tight, and hermetic, and contains at least in part either or both the video processor 632 (discussed below) and the transmitter 608.

Specifically, the controlling unit 508 includes various computing resources described above, including one or more wireless transmitters or transceivers 608 and 660. Among other things, the wireless transmitter and radio frequency (RF) interface 608 transmits processed video as wireless transmitted video 404 to the display devices 124, such as monitors 124A and/or AR devices 124B. In one embodiment, the transmitting antenna 608 for the controlling unit 508 may be fully contained within the controlling unit 508. The wireless transmitter 608 is required to reliably transmit the wireless transmitted video 404 over the distances noted above. In one embodiment, the imaging system 116 complies with electromagnetic compatibility (EMC) requirements specified in EIC60601-1-2.

Among other things, the controlling unit 508 has a controller 604, which provides video receiving, system services, and processing functions, and a video processor 632. The controlling unit 508 may also include the noted wireless transmitter 608, an optional alternate wireless transceiver 660, and the noted user configuration controls 616 (also in FIG. 2).

One challenge associated with powered devices in the sterile field 308 is the limited availability of power sources, as traditional wired power connections are impractical, restrictive, and potentially disruptive in a surgical environment. Sterility requirements prevent the use of standard power cords, as they could introduce contamination risks, obstruct movement, or create hazards for the surgical team. Additionally, wired connections can physically impede the surgeon, restricting their range of motion or interfering with the placement of surgical tools. To solve this, the controlling unit includes an optional power control function 620 and an interface to receive DC power from a power source 612, allowing it to operate independently of external power cables. In preferred embodiments, this DC power is provided by a self-contained battery, eliminating the need for wired connections and ensuring that the device remains fully mobile, sterile, and easily positioned as needed. As such, the video controlling unit 508 may be considered a DC device (e.g., DC powers components of FIG. 5).

Rather than using AC power, the inventors discovered that use of a DC source can be an undesirable way to obtain power in the sterile field 308. For example, batteries may deplete well before the end of a long surgery, which can be dangerous to the patient 112. Devices in the operating room 104 known to the inventors typically are either hardwired to external power sources or positioned outside the sterile zone to avoid contamination risks. For example, surgical equipment requiring power is either placed at a distance or carefully routed with sterile covers over wired connections. This is a key problem the inventors sought to avoid, which can impact the ultimate video displayed on the display device 124. By incorporating a dedicated onboard battery, this device overcomes the challenge of maintaining sterility while delivering reliable power, making it far more practical for direct use in the sterile field 308.

To further enhance convenience, the battery preferably is configured to have a form factor that allows for easy removal and attachment, minimizing disruption when a power swap is necessary. For example, in a puck-shaped controlling unit, the battery could be a cylindrical module that clips securely onto the DC power interface, ensuring stable power delivery while allowing for fast, tool-free replacement. Other potential designs include slide-in battery packs, snap-on modules, or magnetic attachment systems, all of which enable quick battery changes without breaking sterility or requiring extensive handling.

Beyond conventional batteries, additional power solutions could include solid-state batteries, which offer improved safety, longevity, and energy density, or capacitor-based power storage, such as ultracapacitors, which enable rapid charge and discharge cycles. In fact, additional power storage can be onboard, such as capacitors or internal batteries, to maintain power charge during a battery change (e.g., during a long surgical procedure). Another option is wireless inductive charging, which would allow the device to be recharged without direct physical connections, further eliminating the need for wires in or near the sterile field 308. Additionally, a hot-swappable battery system could allow the device to remain powered even during battery changes, preventing downtime and ensuring uninterrupted functionality. By eliminating wired power sources, the device enables greater freedom of movement for the surgical team, maintains a clutter-free workspace, and ensures strict sterility protocols are met, making it a significant improvement over conventional power delivery methods in the sterile field 308.

The power source 612 also may include various forms of external charging/recharging and battery replacement. In one embodiment, the power source 612 may include a primary power source and a backup power source. The power control function 620 may include a battery gauge that measures a current power level from each installed battery. In one embodiment, if the battery gauge determines the power level of the primary power source falls below a threshold, the power control function 620 may automatically switch to the backup power source such that only the backup power source provides device power 624. This may beneficially prevent damage to certain types of primary power sources that may not be discharged below a specified level. In one embodiment, the power control function 620 may provide device power 624 in response to the user configuration controls 616 or a dedicated power ON/OFF control 720.

The wireless transmitter 608 preferably includes a radio frequency (RF) transmitter that transmits processed video 656 to one or more display devices 124, as discussed herein. The processed video 656 may include imaging sensor video 540 that has been processed 656, as also discussed herein. The imaging system 116 may also include the noted optional alternate wireless transceiver 660, which may transmit and receive notifications 412 or other signals or other one-directional or two-directional communication from one or more user devices 408 associated with personnel associated with the operating room 104. The notifications 412 may be related to configuring the imaging system 116, setting or changing operating parameters (e.g., brightness, contrast, sharpness, zoom level, white balance, black level, color correction, image rotation, etc.), error conditions, and power management. It is preferable that this interface 660 be separate from the wireless transmitter 608 for reliability, to reduce interference, and prevent notification data from being displayed on display devices 124 used by the provider 108 to avoid potentially dangerous distractions. Some embodiments, however, may combine both interfaces 608 and 660.

The wireless transmitter 608 and alternate wireless transceiver 660 may use the same or different RF technologies and protocols. However, it may be beneficial to use different RF technologies and protocols to reduce the chance of interference between the RF signals. It is important that the wireless transmitted video 404 be reliably transmitted at a consistent and high level of quality and without interference during the operating procedure. As suggested above, the wireless transmitter 608 and the alternate wireless transceiver 660 may utilize WiFi, WiGig, Bluetooth, ZIGBEE, LoRa, 3G, 4G, 5G, LTE, or any other wireless interface technologies suitable for the bandwidth, bit rate, error corrections, and other requirements.

In one embodiment, the controlling unit 508 may include an embossed “fence” around the power ON/OFF control to reduce the change of accidentally turning the imaging system 116 OFF. In another embodiment, the controlling unit 508 may include a hinged cover over the power ON/OFF control to reduce the change of accidentally turning the imaging system 116 OFF. In another embodiment, the control may only serve as a power ON control and will not power OFF the imaging system 116 even if repeatedly pushed. In that case, powering OFF the imaging system 116 may require either a notification 412 from a user device 408 associated with personnel associated with the operating room 104 or removal of a power source 612 from the controlling unit 508.

The user configuration controls 616 may also include the controls discussed above, such as those to adjust brightness, contrast, sharpness, zoom level, white balance, black level, color correction, or image rotation of the processed video 656. The user configuration controls 616 may also include controls to adjust light source 516 intensity. In one embodiment, when the imaging system 116 is powered OFF, the video processor 632 may save the current states of the various adjustable parameters in a system control 644 area of the memory 636. The next time the imaging system 116 is powered ON, the video processor 632 may retrieve the stored parameters from the system control 644 area and set the adjustable parameters to the retrieved stored values.

The user configuration controls 616 also may include controls that control transmission of processed video 656 or stored video 652. For example, when the imaging system 116 is initially powered ON, this may power all circuits within the device 116 but not automatically initiate transmission of the processed video 656. A separate control may allow the processed video 656 to be transmitted by the wireless transmitter 608. Another control may allow a portion of the stored video 652 (e.g., most recent 10 seconds to be transmitted instead of the live processed video 656). This may allow a provider 108 to quickly replay an event that just occurred and better understand what to do next. As additional control may allow the replayed stored video 652 to be paused on a first activation and resumed at a second activation. In one embodiment, when the replayed portion of the stored video 652 has finished playing, the video processor 632 may resume the usual video processing and transmission of the processed video 656. In one embodiment, the sensor 504 and/or the controlling unit 508 may include an IMU to enable the provider 108 to configure the imaging system 116 with a tap-based user input method.

The controller 604 may include a combination of processing units or processors. In addition, the controller may execute one or more stored programs or applications 648 in one or more accessible memory devices 636.

As known by those in the art, the video processor 632 may act as a specialized hardware component designed to handle the intensive computational tasks required for video encoding, decoding, rendering, and enhancement. It is responsible for processing video signals, improving image quality through features like noise reduction, upscaling, motion compensation, and color correction, and optimizing video playback for various display types. Video processors are commonly integrated into graphics processing units (GPUs), dedicated video processing chips, or standalone video processing cards used in professional video production and broadcasting. While they accelerate specific video-related tasks, they are not typically classified as general-purpose accelerators like GPUs or AI accelerators, though they do offload video workloads from the CPU. Some video processing units (VPUs), found in GPUs, function as hardware accelerators for video compression and playback, making them an essential component in multimedia applications.

To those ends, the video processor 632 may include conventional microprocessors or microcontrollers, application-specific integrated circuits (ASICs), programmable logic devices (PLDs), field-programmable logic devices (FPLDs), field-programmable gate arrays (FPGAS), one or more graphics processing units (GPUs), and/or any combination of hardware, firmware, and/or software. The video processor 632 may include a video streaming engine that provides uninterrupted processed video 656 to the wireless transmitter 608 and/or perform artificial intelligence (AI)-enhanced image processing. The video streaming engine may be either integrated into the video processor 632 or separate from but tightly coupled to the video processor 632. Preferably, the video streaming engine must be able to stream the processed video 656 at a required resolution and frame rate without dropping frames. The video processor 632 may also perform processing to teach a surgical robot how to perform a given surgical operation.

In one embodiment, the memory 636 may include a system control function 644, such as store operating parameters associated with the controller 604, as previously discussed. Additionally, some parameters may be related to hardware capabilities of the imaging system 116, such as power management, memory resources, enabled interfaces, and the like. Power management parameters may include total run time when fully charged, available run time, backup power status, and power management options based on hardware usage.

For example, the controller 604 may be able to power-off certain areas of the memory 636, the alternate wireless transceiver 660 (discussed below), or dim the light source(s) 516 to preserve operating run time. A user device 408 may configure user configuration controls 616 with an expected duration for a surgical operation, including a safety margin. In one embodiment, the memory 636 may include one or more applications 648.

The memory 636 also may store a plurality of applications 648. For example, one application 648 may continuously calculate available runtime for the power source 612 and compare with an elapsed and remaining time for the surgical operation. If the application 648 determines the available run time may reduce the safety margin, the application 648 may turn off hardware devices that do not affect video imaging and transmitting operation—such as unused memory devices 636 and the alternate wireless transceiver 660. If that does not reduce the available run time enough to affect the safety margin, the application 648 may transmit a notification 412 to one or more user devices 408. The notification 412 may include a message that the run time safety margin may still be affected after turning off unused hardware resources and may request a decision to not store video 652 on the device 116 and/or power down a portion of the memory 636 used to store the video 652 or specify a reduced duration of the procedure and/or the safety margin. One goal is to ensure the imaging system 116 can continue to operate at full imaging capability as long as possible. The imaging system 116 user may make the selection in a messaging program (specialized application on the user device 408, a text message, and email, etc.) on the user device 408 and send a reverse notification 412 back to the imaging system 116.

Another application 648 may perform object recognition based on received imaging sensor video 540 to identify what is in the imaging sensor video 540. Yet another application 648 may define the visual boundaries of viewed objects such as surgical instruments, swabs, and anatomical structures. Other applications 648 may apply a marking (e.g., a color, shading, and/or text) to the identified objects in the imaging sensor video 540 or may either a) create a transparent overlay as modified imaging sensor video that includes the markings and b) combines the transparent overlay with the received imaging sensor video 540. Those applications 648 also may add the markings directly to the imaging sensor video 540. In both cases, the processed video 656 may also include any brightness, contrast, sharpness, zoom level, white balance, black level, or image rotation made through the user configuration controls 616 or in notifications 412 received from a user device 408.

The memory device 636 may also include a log (not shown) that correlates objects in the processed video 656 with time stamps. The time stamps may start when the imaging sensor video 540 begins. The log may allow various objects in the processed video 656 to be easily found when reviewing the stored video 652 later. For example, this may allow a reviewing provider 108 to quickly find a portion of the processed video 656 that may include a part of a procedure of particular interest. The log may include notifications 412 transmitted to or received from a user device 408 or received from user configuration controls 616 and a corresponding time stamp.

The controller 604 also may have a light source control function 628. The light source control function 628 may receive control information from the video processor 632 and device power 624 and modulate the device power 624 by the control information. For example, the control information may specify the intensity of the light source 516 should be 70% of a maximum value. The light source control function 628 may modulate the device power 624 to achieve a 70% intensity value in a light source control and power signal 532 to the light source 516. In one embodiment, the modulation may include an amplitude of the light source control and power signal 532. In another embodiment, the modulation may include a frequency of the light source control and power signal 532. Among other things, the modulation may include a duty cycle of the light source control and power signal 532.

As noted above, the sensor 504 may include a IMU sensor 544 to monitor movement and/or position of the sensor 504. The power control function 620 may provide IMU sensor power 548 through the flexible connection to the IMU sensor 544. The video processor 632 may receive the IMU sensor data 552 from the IMU sensor 544 and may execute an application 648 in the memory 636 to determine an exact position of the imaging sensor 512/IMU sensor 544. The IMU sensor data 552 processed by an image recognition application may locate the imaging sensor 512 relative to prior images.

In one embodiment, the controller 604 also may include a transducer, such as a microphone 664. The user configuration controls 616 and potentially a user device 408 application may include a control allowing the imaging system 116 to receive voice commands through the microphone 664 (as noted above). The applications 648 may include a voice recognition application that identifies commands from the received microphone audio and takes actions based on the audio (e.g., “increase light intensity by 10%”). In one embodiment, the voice recognition application 648 may be trained to only accept voice commands from the provider 108. In one embodiment, the video processor 632 may store received microphone audio in the memory and may include time stamps with audio that corresponds to changes in configuration for the imaging system 116.

Accordingly, among other things, the controller 604 can alone or in combination with the video processor actively modify, enhance, and transform the video signal before it reaches the display device 124. It may perform tasks like deinterlacing, frame rate conversion, advanced scaling, noise reduction, HDR tone mapping, and color space conversion. In contrast, the display device 124 primarily receives the processed signal and displays it with minimal adjustments, such as simple scaling, panel response management, and basic color adjustments. The video processor does the primary processing, while the display device 124 serves as the final output device for real-time or near real-time video display.

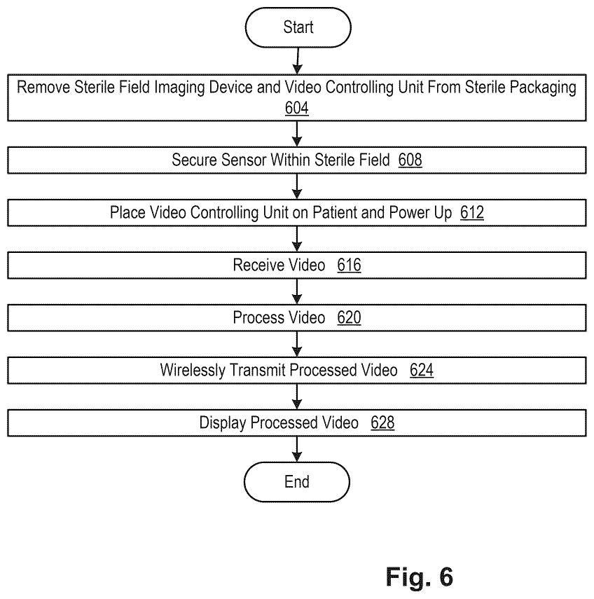

FIG. 6 shows a process of using the imaging system 116 to obtain video or image information in accordance with illustrative embodiments. It should be noted that some of the steps may be performed in a different order than that shown, or at the same time. Those skilled in the art therefore can modify the process as appropriate.

The process begins at block 604, in which the imaging system 116 and the video controlling unit 508 are removed from sterile packaging to be set up in the sterile field 308. Specifically, packaging for sterile surgical devices is designed to maintain sterility until the moment of use while providing ease of access in a sterile field 308. These packages may be made of materials with high barrier properties to prevent microbial contamination, such as Tyvek® (a breathable, medical-grade polyethylene fiber), medical-grade paper, and multi-layer plastic films. The packaging often features a combination of rigid trays, pouches, or blister packs sealed with heat or adhesives to ensure integrity. Additionally, sterile device packaging may include visual indicators such as sterility indicator strips and tamper-evident seals to confirm sterility before use.

One design feature of surgical device packaging is its ability to be opened aseptically, ensuring that the contents remain sterile when transferred into the sterile field 308. Peel pouches and header bags, for example, allow easy opening without contaminating the device inside. Rigid trays with snap-fit or Tyvek® lids provide robust protection for delicate instruments like surgical scissors or orthopedic implants. Double-wrapped packaging, commonly used for surgical instruments and implantable devices, allows for a secondary sterile barrier when transferring between environments, such as from storage to the operating table.

Examples of sterile surgical device packaging include double-pouched Tyvek® and medical grade polyethylene pouches, rigid thermoformed plastic trays with a Tyvek® lid, or foil laminated barrier pouch with desiccant and shock absorption. One relevant feature is for the packaging to have electrostatic protection to prevent component failure.

These packaging solutions are designed to meet stringent regulatory requirements such as FDA guidelines to ensure patient safety and efficacy in surgical environments.

In other embodiments, these components are sterilizable for use in the sterile field 308. For example, they may be sterilized inside or outside the sterile field 308. For example, these devices can be configured to be “ready to sterilize” in the operating room 104 using low-temperature hydrogen peroxide plasma sterilization, which effectively eliminates microbes without damaging sensitive electronics. The process involves pre-cleaning the device with a sterile wipe and a non-corrosive disinfectant, then placing it inside a compatible sterilization pouch or container. The device is loaded into the sterilizer, where vaporized hydrogen peroxide is introduced and converted into plasma, destroying microorganisms while ensuring no harmful residue remains. After the cycle is complete, the device is ready for aseptic presentation in the sterile field 308, making it a suitable method for delicate medical electronics.

For the devices can be configured and constructed can withstand low-temperature hydrogen peroxide plasma sterilization, the design should be formed from materials and construction techniques that ensure durability, sterility, and resistance to degradation. To that end, in preferred embodiments, the housing is made from medical-grade materials such as stainless steel, titanium, anodized aluminum, polycarbonate, or PEEK (polyether ether ketone), all of which can endure hydrogen peroxide exposure without breaking down. The device also preferably is fully sealed to prevent vapor penetration, such as with an IP67 or higher rating. Hermetic sealing techniques, such as laser welding, ultrasonic bonding, or the use of sterilization-resistant O-rings (e.g., silicone, PTFE, or EPDM), also can help protect internal electronics.

Internally, electronics may be designed to resist chemical exposure and moisture by using conformal coatings like parylene or silicone encapsulation to shield circuit boards. Components such as batteries, sensors, and connectors should be chosen for their ability to withstand sterilization without performance degradation. Venting holes or fans should be avoided, as they could allow hydrogen peroxide ingress. Instead of adhesives that may degrade, mechanical fasteners and welded seams can be prioritized. Additionally, LCD or OLED screens (e.g., on the video controlling unit 508), if required, can be covered with durable materials like sapphire glass or reinforced polycarbonate. By avoiding porous materials, heat-sensitive adhesives, and ventilation-dependent designs, the device can be reliably sterilized and safely introduced into a sterile surgical field.

At block 608, the sensor 504 is secured within the sterile field 308. Securing the sensor 504 may involve attaching the imaging head to a stationary point such as a retractor within the sterile field 308. This preferably keeps the sensor 504 stationary to facilitate clear imaging sensor video 540. In one embodiment, different adapters 712 may allow the sensor 504 to be secured to different stationary points. For example, some retractors may include a dovetailed channel that allows other items to couple to the retractor. Flow proceeds to block 612.

At block 612, the video controlling unit 508 is placed on the patient 112. As noted, this placement does not cause trauma to the patient 112. To that end, as noted above, the housing preferably has a patient contact surface formed from a biocompatible material and sized to be supported on the patient's body in the sterile field 308. The video controlling unit 508 preferably remains there even when imaging the patient 112.

At this point, the sensor 504 and video controlling unit 508 are powered to an ON state. In one embodiment, the power ON state is received by the power control function 620, which routes DC device power 624 to the video processor 632, the memory 636, the wireless transmitter 608, and to imaging sensor power 536, light source control and power 532, and IMU sensor power 548. Flow proceeds to block 616.

At block 616, the sensor 504 produces images with its imaging sensor 512. Specifically, in response to receiving imaging sensor power 536, the imaging sensor 512 produces imaging sensor video 540 corresponding to the imaged area 528 to the video processor 632 of the video controlling unit 508. The received video 540 may depend on a projected light intensity of the illuminated area 524. In one embodiment, the light intensity of the illuminated area 524 (i.e., intensity of the light source 516) following power ON may be determined by a stored value in the system control 644 portion of the memory 636. For example, when the video processor 632 receives a power OFF control indication from the power ON/OFF control 720, the video processor 632 may store a current value of the light intensity to the system control 644 area of the memory 636. Flow proceeds to block 620.

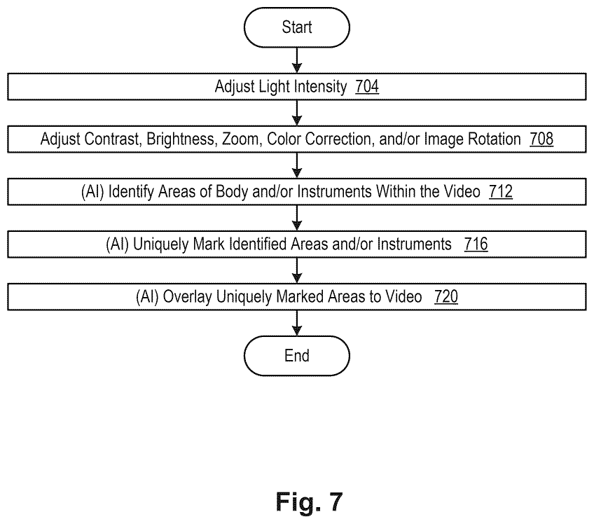

At block 620, the video processor 632 of the video controlling unit 508 processes the imaging sensor video 540. Processing the imaging sensor video is described in more detail above and with respect to FIG. 7. Flow proceeds to block 624.

At block 624, the imaging system 116 wirelessly transmits the processed video 656 to one or more display devices 124, such as video monitor(s) 124A and/or AR device 124B. In one embodiment, the video processor 632 stores the imaging sensor video 540 as stored video 652 in the memory 636. The video processor 632 may then process the stored video 652 in one or more ways. In one embodiment, the video processor 632 may process the imaging sensor video 540 as it is stored and stream the processed video to the wireless transmitter 608. In another embodiment, the video processor 632 may store the video 540 to stored video 652 in an unprocessed state while simultaneously processing and streaming the video to the wireless transmitter 608. The wireless transmitter 608 wirelessly transmits video 404 to display devices 124. Flow proceeds to block 628.

At block 628, video monitor(s) 124 and/or an AR device 124B displays the wireless transmitted video 404, and the provider 108 sees the imaged area 528 in real time. Flow ends at block 628.

FIG. 7 shows a video processing process for the imaging system 116 in accordance with illustrative embodiments. Much of this is performed in step 820 of FIG. 6. It should be noted that some of the steps may be performed in a different order than that shown, or at the same time. Those skilled in the art therefore can modify the process as appropriate.

The process begins at block 704, in which the light intensity of the illuminated area 524 may be adjusted. Although this is not “processing video” as discussed elsewhere with respect to FIG. 6, the light intensity of the illuminated area 524 may have the effect on the quality of the imaging sensor video 540. The light intensity may be locally or remotely adjusted, as described herein.

In one embodiment, the video processor 632 may be configured to output multiple video streams at different resolutions and compression ratios, for example, a full resolution, uncompressed stream for a surgical display, and a reduced-resolution compressed stream to a tablet or mobile computing device for recording or offloading to an electronic medical record (EMR) system. The video processor 632 may also be configured to provide different overlays (text, graphics) to the different video streams. Other embodiments minimize or have no compression. Flow proceeds to block 708.

At block 708, the contrast, brightness, sharpness, white balance, black level, zoom, color correction, and/or image rotation may be performed by the video processor 632. These parameters may be either locally or remotely changed or adjusted, as discussed herein. Brightness is an attribute of visual perception in which a source appears to be radiating or reflecting light. In other words, brightness is a perception elicited by the luminance of a visual target. Contrast is the difference in luminance or color that makes an object (or its representation in an image or display) visible on a background of different luminance or color. Sharpness is related to the contrast of edges within an image. White balance is related to the color temperature of a color image. Black level is the brightness value of the darkest part of an image. Zoom is the process of enlarging or reducing the apparent size, not physical size, of something visual. This enlargement or reduction is quantified by a size ratio called optical magnification.

Color correction is a process used in digital imaging and other disciplines, which uses filters to alter the overall color of the light. Typically, the light color is measured on a scale known as color temperature, as well as along a green-magenta axis orthogonal to the color temperature axis. Image rotation is a process of rotating an image either clockwise or counterclockwise a certain amount. Image rotation may be expressed in increments of 90 degrees. For example, depending on the position and orientation of the sensor 504 (and hence the imaging sensor 512 captured within the sensor 504), it may be helpful to rotate the image to display the image in a way that makes the most sense to the provider 108. Flow proceeds to block 712.

At blocks 712-720, various applications 648 may further process the video to add helpful information for the provider 108, such as pointing out structures within the body and/or surgical instruments within the field of view of the video. In one embodiment, the processing steps described with respect to blocks 712-716 are performed after the processing steps discussed with respect to blocks 704-708. In another embodiment, the processing steps described with respect to blocks 712-716 are performed before the processing steps discussed with respect to blocks 704-708. The steps of blocks 712-720 may be performed by one or more applications that may employ large language models, neural networks, and/or techniques using artificial intelligence (AI).

At block 712, the video processor 632 may execute a stored application 648 that identifies areas of the body and/or instruments within the video. Areas of the body may include tissue masses, organs, vascular or arterial items, bones, cartilage, holes, and the like. Surgical instruments may include clamps, retractors, pins, scalpels, and the like. In one embodiment, the stored application 648 may utilize object recognition and include many data structures that the application uses to identify areas of the video. Flow proceeds to block 716.

At block 716, the video processor 632 may execute a same or different stored application 648 to mark the identified areas or instruments. For example, the application 648 may mark different areas by color/shading or similar areas by a same color/shading. In one embodiment, the marked areas may include text or alphanumeric values. In one embodiment, the video processor 632 may share the processing and overlay generation tasks between an application 648 and an application executing in a suitably enabled mixed reality wearable display device 124B. Flow proceeds to block 720.

At block 720, the video processor 632 may overlay uniquely marked areas to the video. The overlaid marked areas may be presented as a transparency layer over the video. For example, a transparent red color over arterial tissue or a blue color over veinous tissue in the video. At this point the processed video 656 may be streamed or transmitted to the display devices 124 as wireless transmitted video 404. In some embodiments, the processed video 656 may be stored as stored video 652. In one embodiment, the video processor 632 may utilize information from non-visible light imaging to compute video overlays. The video processor 632 may use information received from other systems, such as navigation systems, fluoroscopy, X-Ray, ultrasound, and vital signs monitors, to compute visual overlays. Flow ends at block 720.

Various embodiments of the invention may be implemented at least in part in any conventional computer programming language. For example, some embodiments may be implemented in a procedural programming language (e.g., “C”), or in an object-oriented programming language (e.g., “C++”). Other embodiments of the invention may be implemented as a pre-configured, stand-alone hardware element and/or as preprogrammed hardware elements (e.g., application specific integrated circuits, FPGAS, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in any of a variety of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

The embodiments of the invention described above are intended to be merely exemplary; numerous variations and modifications will be apparent to those skilled in the art. Such variations and modifications are intended to be within the scope of the present invention as defined by any of the appended claims.

What is claimed is:

1. A surgical imaging system for communicating images from an imaging sensor to a display device within an operating room, the surgical imaging system comprising:
a video controlling unit configured to receive the images from the imaging sensor, the video controlling unit being sterile or sterilizable sufficient for use in the sterile field of an operating room, the video controlling unit comprising:
a video processor configured to alter the received images to produce processed images from the received images, the video processor configured to produce the processed images in a protocol that is compatible with the display device for real-time or near real-time display,
a transmitter configured to wirelessly and substantially lag-free transmit the processed images from the sterile field to the display device, the transmitter configured to transmit the processed images at a frame rate compatible with a frame rate displayable by the display device, the transmitter configured to transmit the processed images with an effective radiated power of 1-100 milliwatts,
a DC interface configured to receive direct current power to power the transmitter and the video processor; and
a housing containing at least in part either or both the video processor and the transmitter, the housing having at least one patient contact surface formed from a biocompatible material and sized to be supported on the patient's body in the sterile field when imaging with the imaging sensor,
the video controlling unit configured to cause no trauma to the patient when supported on the patient's body.

2. The system as defined by claim 1 wherein the protocol comprises High-Definition Multimedia Interface.

3. The system as defined by claim 1 further comprising the display device.

4. The system as defined by claim 1 wherein the display device is outside of the sterile field and within the operating room.

5. The system as defined by claim 1 further comprising the imaging sensor, the imaging sensor comprising a camera using one or more of IR, visible, UV and ICG wavelengths.

6. The system as defined by claim 5 further comprising an inertial measurement unit coupled with the imaging sensor and producing one or more of angular velocity, acceleration, position, and orientation signal relating to inertial activity of the sensor.

7. The system as defined by claim 1 wherein the video controlling unit is sized to have a low profile to avoid obstructing surgical access when on the patient's body.

8. The system as defined by claim 1 wherein the video controlling unit comprises an input for receiving input information from a user, the video controlling unit being controlled at least in part by the received input information.

9. The system as defined by claim 7 wherein the input comprises a tactile input, an input configured to receive voice commands, or an input configured to receive gesture commands.

10. The system as defined by claim 1 wherein the display device comprises smart goggles configured to be worn across the eyes of a user in sterile field, the display device having an input to wirelessly receive the processed images.

11. The system as defined by claim 1 wherein the video processor is configured to alter the received images by changing one or more of brightness, contrast, sharpness, white balance, black level, zoom level, apply color correction, or rotation.

12. The system as defined by claim 1 wherein the video controlling unit is sealed in an air-tight package having a sanitized interior.

13. The system as defined by claim 1 wherein the housing is sealed to have a liquid resistant interior containing the video processor.

14. The system as defined by claim 1 further comprising computer program product for use on a computer system, the computer program product comprising a tangible, non-transient computer usable medium having computer readable program code thereon, the computer readable program code comprising program code for controlling the video controlling unit.

15. The system as defined by claim 1 wherein the video controlling unit has a length, width, and height, the length being 3 to 9 inches, the width being 3 to 14 inches, and the height being 3 to 8 inches, the video controlling unit having a weight of 8 to 32 ounces.

16. A surgical imaging system for communicating images from an imaging sensor to a display device within an operating room, the display device configured to communicate and operate in accordance with a display protocol, the surgical imaging system comprising:
a battery powered video controlling unit configured to receive the images from the imaging sensor, the video controlling unit being sterile sufficient for use in the sterile field of an operating room, the video controlling unit comprising:
a video processor configured to alter the received images to produce processed images from the received images, the video processor configured to produce the processed images in the display protocol,
a transmitter configured to wirelessly and substantially lag-free transmit the processed images from the sterile field to the display device for real-time display or near real-time display, the transmitter configured to transmit the processed images with the display protocol toward the display device, the transmitter configured to transmit the processed images to the display device spaced at or between about 0.5 meters to 50 meters away from the transmitter, and
a housing containing at least in part one or more of the video processor and the transmitter, the housing having at least one patient contact surface formed from a biocompatible material and sized to be supported on the patient's body in the sterile field when imaging with the imaging sensor, the video controlling unit configured to cause no trauma to the patient when supported on the patient's body; and
an input for receiving input information from a user via one or more of user voice, user gesture, and direct user contact, the video controlling unit being controlled at least in part by the received input information.

17. The system as defined by claim 16 wherein the protocol comprises High-Definition Multimedia Interface.

18. The system as defined by claim 16 wherein the display device is outside of the sterile field and within the operating room.

19. The system as defined by claim 16 wherein the controlling unit is sized to have a low profile to avoid obstructing surgical access when on the patient's body.

20. The system as defined by claim 16 wherein the display device comprises eyewear configured to be worn across the eyes of a user in sterile field, the display device having an input to wirelessly receive the processed images.

21. The system as defined by claim 16 wherein the video controlling unit is sealed in a package having a sanitized interior.

22. The system as defined by claim 16 wherein the housing is sealed to have a liquid resistant interior containing the video processor.

23. The system as defined by claim 16 further comprising computer program product for use on a computer system, the computer program product comprising a tangible, non-transient computer usable medium having computer readable program code thereon, the computer readable program code comprising program code for controlling the video controlling unit.

24. A method of imaging during a surgical procedure for a display on a display device that communicates and/or operates in accordance with a display protocol, the surgical procedure being in an operating room having a surgical field, the method comprising:
positioning an imaging sensor relative to a patient in the surgical field to produce a plurality of images of the patient during the surgical procedure, the patient and imaging sensor being in the sterile field of the operating room;
imaging the patient during the surgical procedure using the imaging sensor;
placing a sterile video controlling unit directly on the patient during imaging, the patient's body supporting the video controlling unit during the surgical procedure without causing trauma to the patient's body;
powering the video controlling unit in the sterile field with DC power either before or after positioning on the patient's body;
receiving, by the video controlling unit, images generated by the imaging sensor;
altering the received images, by the video controlling unit, to produce processed images from the received images, the processed images being in the display protocol;
transmitting, wirelessly, substantially lag-free, and with an effective radiated power of 1-100 milliwatts, the processed images from the sterile field to the display device at a frame rate compatible with a frame rate displayable by the display device; and
displaying, by the display device, the processed images in real-time or near real-time.

25. The method as defined by claim 24 further comprising receiving input information from a user via one or more of user voice, user gesture, and direct user contact, the video controlling unit being controlled at least in part by the received input information.

26. The method as defined by claim 24 wherein the display device comprises eyewear.

27. The method as defined by claim 24 wherein further comprising controlling the video processor to alter the received images by changing one or more of brightness, contrast, sharpness, white balance, black level, zoom level, apply color correction, or rotation.

28. The method as defined by claim 24 further comprising removing the video controlling unit from a sterile package before placing.

29. The method as defined by claim 24 further comprising displaying a control interface on a handheld computer device, the method further using the control interface to control display on the display device.

30. The method as defined by claim 24 wherein transmitting comprises transmitting the processed images to the display device at a power level sufficient to reach the display device when spaced at or between about 0.5 meters to 50 meters away from the video controlling unit.
